(19) United States
(12) Patent Application Publication
(10) Pub. No.: US 2023/0146654 A1
(43) Pub. Date: May 11, 2023

(54) ELASTIC DIAPER ELEMENT

(71) Applicant: RKW SE, Mannheim (DE)

(72) Inventor: Christoph WILLING, Vreden (DE)

(73) Assignee: RKW SE, Mannheim (DE)

(21) Appl. No.: 17/802,575

(22) PCT Filed: Oct. 29, 2020
(86) PCT No.: PCT/EP2020/080352

§ 371 Date: Aug. 26, 2022

(30) Foreign Application Priority Data
Feb. 28, 2020 (DE) 102020105263.4

(51) Int. Cl. A61F 13/15

(52) U.S. Cl. CPC A61F 13/15699; A61F 2013/49023

(57) ABSTRACT

An elastic diaper element that includes an elastic layer (1) and at least one layer (2) made of a nonwoven. The elastic element has connection regions (5) between the elastic layer (1) and the non-woven layer (2). The connection regions (5) extend in a preferred direction. The non-woven layer (2) is wavy when the diaper element is in the unstretched condition in order to provide regions (6) as a reserve allowing stretching. The connection regions (5) have an oscillating shape along their extent.

### TECHNICAL FIELD

The invention relates to an elastic diaper element having an elastic layer and a layer from a non-woven.

Elastic elements are used in diapers in order to guarantee a good fit and tightness. The diaper element according to the invention is preferably used as a waist band. The material is likewise particularly suitable for use as an elastic closure element, i.e. a so-called “back ear”, on baby diapers.

### BACKGROUND

DE 602 04 588 T2 describes a production method for an elongatable elastic composite material. Here, rollers which have a multiplicity of axially spaced-apart teeth of identical shape that lie next to one another and run in the circumferential direction are used. The spacings between teeth that lie next to one another form depressed flutes of identical configuration that run in the circumferential direction.

A diaper having an upper and a lower layer is described in DE 689 23 866 T2. An elastic band is fastened to the elastic layer in the non-tensioned state. The elastic band is connected to the elastic layer across the entire area. Gathered regions are formed once the elastic band has been elongated and relaxed.

EP 217032 B1 relates to a laminate having an elastic material which at mutually spaced apart locations is connected to at least one web to be pleated. The elastic material is an elastic fibrous web that is not wavy.

A method for producing a wavy elastic laminate is described in EP 1 807 035 B1. Here, an elastic compound in a molten state is applied to a carrier web in order to form an elastic element. A preform of an elastic composite material is formed by elongating the carrier web. Stretching of the preform takes place thereafter. A substrate is connected to the stretched preform so as to form a wavy elastic laminate once the stretched preform has relaxed.

EP 2 024 178 B1 describes a method for producing an elastically elongatable laminate having three layers. The laminate comprises an elastic film and two layers from a non-elastic non-woven fabric. A non-woven crepe is used in one variant. A first elastic laminate in an elongated state is connected to a non-elastic non-woven layer.

### SUMMARY

It is an object of the present invention to provide a diaper element which has favorable elongation properties and at the same time guarantees a high tear strength. When stressed with a force, the element should behave in an elastic manner but also build up sufficient resistance so as to impart to the consumer a feeling of a high-quality product. When used as a “back ear”, the element must not tear because the diaper otherwise can no longer be closed. At least one non-woven layer herein, by way of a wavy configuration, should provide a sufficient but not excessive reserve for stretching the element. The laminate should not pose any health risk and be ecologically sustainable. Moreover, no odors should emanate from the product. Furthermore, the product should be pleasantly tactile. Moreover, an optimal fit of the diaper on the body should be guaranteed by the diaper element so that cavities between the diaper and the body are avoided and leakages of liquid are prevented.

This object is guaranteed according to the invention by a diaper element, a method and the use thereof having one or more of the features described herein. Preferred variants can be derived from the claims, the description, the exemplary embodiment and the drawings.

The diaper element according to the invention comprises a layer from an elastic material. The elastic layer is provided with a layer from a non-woven material at least on one side. Such non-woven materials typically have a limited elongating capability. Therefore, a non-woven layer in the waist band elements according to the invention is connected to the elastic layer in a wavy shape. The troughs of the waves are connected to the elastic layer, while the peaks of the waves protrude away from the elastic layer and enclose a cavity toward the elastic layer. As a result of the waves, the non-woven layer in the unstretched state forms a reserve for enabling stretching of the element.

The elastic element has connection regions between the elastic layer and the layer from a non-woven, and the connection regions have an extent in a preferred direction, wherein the layer from a non-woven in the unstretched state of the diaper element is wavy so as to guarantee regions as a reserve for enabling stretching.

According to the invention, the connection regions extend in an oscillating manner between the elastic layer and the layer from a non-woven of a diaper element. In comparison to connection regions that run in a rectilinear manner, the elongation properties, the tear strength as well as the haptics are improved as a result.

It has proven particularly favorable for the connection regions to have a wavy, in particular sinusoidal, alignment. The profile here extends about an imaginary auxiliary line, in that the amplitude within one phase length once runs above and once below the imaginary auxiliary line.

In an advantageous alternative of the invention, the connection regions have a zigzag alignment. The connection regions here can be configured as sawtooth-shaped or triangular lines. These lines have straight sections and change the direction of their alignment by way of defined angles.

It has proven particularly favorable for the connection regions to have an extent about the imaginary auxiliary line thereof that is perpendicular to the tensile direction of the diaper element. In the wavy profile, the angle between the gradient tangent of the connection region and the direction of the tensile stress continually varies in the range from 45° to 135°. This results in a pattern of regions of different elongations, as a result of which the elastic reserve and at the same time the restoring force are significantly increased.

In one variant of the invention, the connection regions which are embodied as oscillating lines preferably have identical spacings.

It has proven favorable here for the spacing of these lines to be more than 1 mm, preferably more than 2 mm, in particular more than 2.5 mm, and/or less than 8 mm, preferably less than 7 mm, in particular less than 6 mm.

In an alternative variant, the oscillating lines of the connection region are mutually disposed so as to be mirror-symmetrical. According to the invention, small reserve regions alternate with large reserve regions for stretching as a result, due to which the elongation capability in particularly stressed regions of the diaper element is increased. It has proven favorable here for the spacing of these lines to be more than 1 mm, preferably more than 1.5 mm, in particular more than 2 mm, and/or less than 16 mm, preferably less than 10 mm, in particular less than 6 mm.

In a further alternative, the oscillating lines of the connection regions are displaced in terms of the phases thereof, this resulting in randomly distributed small and large reserve regions for stretching. The same effect for increasing the elongation capability is achieved when the oscillating lines of the connection regions differ in terms of their amplitude as well as their phase length.

A variant in which segments on connection regions alternate with sections on interruptions is also conceivable. Formed herein are rows having segments of connection regions and segments of interruptions that are disposed directly next to one another in a mutually offset manner.

A method in which the wavy non-woven layer is impressed in the fusible elastic layer using a roller that has elevations will preferably be used for producing the elastic laminate. According to the invention, a roller of which the elevations are configured so as to be wavy is used in the process.

Alternatively, a materially integral connection by means of ultrasonic welding is established between the non-woven layer and the elastic layer so as to produce the connection regions. To this end, the non-woven layer is imparted a wavy shape by rolling, and said non-woven layer in the wave troughs is welded to the elastic layer by way of an oscillating profile.

The use of rollers having webs that oscillate in a mutually parallel manner has proven particularly favorable. The oscillating profile of the webs here may be configured so as to be wavy, in particular sinusoidal or in the shape of saw teeth or triangular teeth, said oscillating profile being distinguished by an oscillating section that is repeatedly mirrored. The profile of the webs is fundamental to the profile of the connection regions between the elastic layer and the layer from a non-woven.

In a further variant of the method, rollers having parallel and rectilinear flutes are used. In order for an oscillating profile of the connection regions to be implemented, at least two rollers are set in a mutually oscillation motion. The rollers here oscillate in such a manner that one phase has a length of more than 10 mm, preferably more than 20 mm, in particular more than 30 mm, and/or less than 200 mm, preferably less than 150 mm, in particular less than 100 mm, and an amplitude of more than 0.5 mm preferably more than 0.8 mm, in particular more than 1.0 mm, and/or less than 10 mm, preferably less than 8.0 mm, in particular less than 6.0 mm.

In one variant of the invention, the wavy profile of the non-woven layer can be formed between two rollers that have elevations and depressions, wherein at least one of the two rollers is configured as a fluted roller. The elevations of the one roller herein engage in the depressions of the other roller and vice versa. According to the invention, the elevations have an oscillating profile. In another variant of the invention, the elevations have a zigzag fluted profile, in particular in the shape of a saw tooth or a triangular tooth.

Alternatively, the oscillating profile of the connection regions can be generated by ultrasonic welding in a wavy and/or jagged profile.

The terms “non-woven” or “non-woven fabric”, respectively, refer to a fabric which, without weaving or knitting, can be produced from continuous filaments and/or discontinuous filaments by means of methods such a spun-bonding, carding or melt-blowing. The non-woven fabric can comprise one or a plurality of non-woven layers, wherein each layer may contain continuous filaments or discontinuous filaments. A non-woven fabric can also comprise bi-component fibers which can display fiber structures such as, for example, shell/core, side-by-side structures.

The term “elastic” preferably refers to any material which, upon applying a directed force, can be stretched to an elongated length of at least approximately 160% of the relaxed, original length thereof without tearing or breaking, and which, upon removing the applied force, is restored by at least approximately 55% of the extended length thereof, preferably restored substantially to the original length thereof, that is to say that the restored length is less than approximately 120%, preferably less than approximately 110%, more preferably less than approximately 105%, of the relaxed original length.

The term “diaper” preferably refers to single-use absorbent products which absorb and enclose fluids. The term comprises inter alia diapers with closures, diaper pants, training pants, swimming diapers, incontinence products for adults, and the like.

In the production of the diaper element, prior to the connecting step, the non-woven layer is brought to a three-dimensional, wavy shaped in that said non-woven layer is guided across a special device. This device may be a roller that has elevations and configures the wavy profile of the non-woven layer as a result. Additionally or alternatively, prior to the connecting step, the non-woven layer can be guided across an element which in an arcuate manner extends so far as the extruded elastic layer, so that the wavy profile of the non-woven layer is preserved up to the connecting step. The element can be configured as a finger rail, for example.

The ratio between the raised and the depressed regions of the devices that ae used for forming the wavy profile and in the connecting step is important for an optimal waist band laminate. This ratio is also referred to as the web-to-groove ratio. This web-to-groove ration is preferably less than 1:1, preferably less than 1:2.

It has proven particularly favorable for the connection regions to have a width of more than 0.1 mm, preferably more than 0.3 mm, in particular of more than 0.5 mm, and/or a width of less than 1.5 mm, preferably less than 1.3 mm, in particular less than 0.9 mm.

Regions in which the wavy non-woven layer by way of the elevations thereof protrudes in a wavy shape away from the elastic layer and encloses cavities are disposed between the connection regions. These regions serve as a reserve for stretching of the diaper element, wherein there is no connection present between the non-woven layer and the elastic layer in these regions.

In terms of the overall area of the flat film, the reserve regions have a significantly larger proportion than the connection regions. The proportion of the reserve regions is preferably more than 60%, in particular more than 70%, preferably more than 80%, of the overall area. The surface of the solidified flat elastic layer is used as the reference overall area.

In the reserve regions, there is no connection present between the non-woven layer and the elastic layer.

As a result of the wavy design, the non-woven layer of a laminate portion is significantly longer than the elastic layer. The non-woven layer is preferably longer than the elastic layer by a factor of more than 1.5, in particular by a factor of more than 2.0, preferably by a factor of more than 2.5.

In one preferred variant of the invention, stretching of the laminate in the transverse direction takes place after the connecting procedure. The elongation preferably takes place so as to be below the elongation at break of the non-woven layer.

It has proven particularly favorable for the non-connection regions, thus the reserve regions, to have a width of more than 1.5 mm, preferably more than 2 mm, in particular more than 2.5 mm, and/or a width of less than 6 mm, preferably less than 5 mm, in particular less than 4 mm.

The connection regions preferably have a width of more than 0.1 mm, in particular more than 0.2 mm, preferably more than 0.3 mm, and/or a width of less than 0.1 mm, preferably less than 0.8 mm, preferably less than 0.6 mm.

The reserve regions, or connection regions, respectively, are preferably configured in the shape of strips than run transversely to the tensile direction of the waist band.

In order for the connection regions to be achieved, rollers having a surface structure are preferably used, wherein the height of the elevations is more than 100 μm, preferably more than 500 μm, in particular more than 1 mm, and/or less than 12 mm, preferably less than 10 mm, in particular less than 8 mm.

The non-woven layer is preferably composed of a hydroentangled non-woven material. Fibers in the non-woven material can be re-oriented by hydroentanglement such that the original two-dimensional fiber alignment is transformed to a three-dimensional fiber orientation. The fibers are more intensely incorporated in the non-woven fabric. This non-woven layer preferably has a specific weight of 5 to 80 g/m², preferably of 10 to 70 g/m², in particular of 15 to 35 g/m².

The hydroentangled non-woven layer is preferably composed of non-woven material from continuous filaments. The latter, by virtue of the production process thereof, offer a fibrous pile which is preferably configured in the manner of loops.

Spinning-capable polymers such as, for example, polyester, PLA, polyolefins, in particular polypropylene and polyethylene, can be used as materials for producing the continuous filaments.

The use according to the invention of hydroentangled non-woven material as the wavy non-woven layer that forms reserve regions for elongating the diaper element is particularly advantageous. As a result of the wavy formation, the hydroentangled non-woven material is deformed in such a manner that the fibers in the connection regions are stretched and as a result are preferably imparted an orientation. As a result, particularly advantageous connection zones in which the fusible material encloses the elongated and aligned filaments of the hydroentangled non-woven material are achieved, and a particularly favorable form-fitting composite is created upon solidification. It has been surprisingly demonstrated that a diaper element having particularly favorable properties is achieved when using a hydroentangled non-woven material. The non-woven layer from the hydroentangled non-woven material can be particularly readily deformed. Practically no width is lost in the infed non-woven web during the production process, despite the formation of waves.

The elastic layer is preferably a polypropylene and/or a polyethylene block copolymer. The elastic film preferably has a specific weight of 5 to 140 g/m², in particular of 10 to 130 g/m², preferably of 20 to 40 g/m².

Alternatively, the elastic layer may also be composed of SBC (styrene block copolymer) or an elastic polyurethane.

In one variant of the invention, the elastic layer is constructed in multiple layers and preferably embodied as a co-extruded film. The latter in one advantageous variant comprises a core layer, and a skin layer which is significantly thinner in comparison. The skin layer preferably has a specific weight of less than 5 g/m², in particular less than 4 g/m², preferably of less than 3 g/m², and/or more than 0.3 g/m², in particular more than 0.6 g/m², preferably more than 0.9 g/m².

In one variant the core layer is embedded between two external skin layers.

The core layer is preferably composed of an elastic polyolefin or an SBC (styrene block copolymer) or a polyurethane.

The skin layer is preferably composed of a polyethylene, a polypropylene or an EVA (ethylene vinyl acetate copolymer).

In one favorable variant of the invention, filled polyolefins are used as the skin layer. Mineral materials such as calcium carbonate or talc are used as filler materials, for example. The filler material proportion is preferably more than 20% by weight, in particular more than 30% by weight, preferably more than 40% by weight.

Blends may also be used for forming the skin layer. For example, blends of polyolefins and polystyrene and/or blends of polyolefins and PLA are suitable here. A filler material is not mandatory in such blends.

The skin layer is designed such that unblocking from the sticky core layer is guaranteed. Moreover, the skin layer is easily deformable and readily elongatable.

In one variant of the invention, the diaper element comprises a non-woven layer from a carded non-woven material. The carded non-woven material used is preferably composed of polypropylene fibers and/or of mixtures of different types of fibers such as, for example polypropylene/viscose, polypropylene/polyamide, polypropylene/polyester, etc. The carded non-woven material can also be composed of polypropylene and/or polyethylene copolymer. The specific area of weight of the carded non-woven material is preferably 10 to 40 g/m², in particular between 15 and 25 g/m². The carded non-woven material can be solidified by means of a calendar, for example, and/or by means of air and/or a water jet acting thereon.

The elastic layer in the diaper element can also be embedded between two layers of a non-woven. At least one layer herein is preferably composed of a hydroentangled non-woven material. The second non-woven layer can be composed either of a hydroentangled non-woven material or a carded non-woven material or a spunbonded non-woven material. The second non-woven layer can either be configured so as to be wavy, or have a flat profile.

As a result of the design of the diaper element according to the invention, the diaper bears in an optimum manner on the body and guarantees an optimum fit. As a result of the arrangement of the folds thereof, the diaper element has a voluminous design and fills cavities between the diaper and the body so that any leakage is effectively prevented.

The laminate according to the invention preferably has straight pleats on the non-woven material surface and possesses a positive restoring elasticity. The transverse elasticity and plastic deformation upon impingement with stretching, also referred to as the “set”, of laminates which are compressed with webs in the longitudinal direction displays a proportional correlation with the composite area. The composite area in the laminate according to the invention is decreased in size. At the same time, sufficient inter-composite adhesion is maintained here. The inter-composite addition of the laminate according to the invention in the machine direction (MD) is preferably more than 1N/25 mm while simultaneously improving the elasticity in the transverse direction (CD).

Both requirements are achieved by reducing the web compression face of the rollers used. In one variant of the invention, the webs of the rollers are provided with machined cuts, thus are interrupted.

Rollers having parallel webs and flutes with an oscillating profile are preferably used here. Wavy fluted rollers of identical circumference, having webs and flutes, have proven to be particularly advantageous. One phase of the oscillating profile of these flutes has a length of more than 10 mm, preferably more than 20 mm, in particular more than 30 mm, and/or less than 200 mm, preferably less than 150 mm, in particular less than 100 mm. One phase of the oscillating profile of these flutes has an amplitude of more than 0.5 mm, preferably more than 0.8 mm, in particular more than 1.0 mm, and/or less than 10 mm, preferably less than 8.0 mm, in particular less than 6.0 mm. The rollers, provided with wavy flutes to a depth of approx. 0.5 to 10 mm, engage in one another and, by way of an identical circumference and speed, roll on one another in a mutually engaging manner.

### BRIEF DESCRIPTION OF THE DRAWINGS

Further advantages and features of the invention are derived from the description of an exemplary embodiment by means of drawings, and from the drawings per se.

In the drawings:

FIG. 1 shows a section through a diaper element according to the invention;

FIG. 2 shows a schematic plan view of the diaper element, having a first variant of the wavy connection regions;

FIG. 3 shows a schematic plan view of the diaper element, having a second variant of the wavy connection regions;

FIG. 4 shows a schematic plan view of a connection region extending in a wave-shaped manner, highlighting the continuously varying angle between the connection region and the tensile direction;

FIG. 5 shows a schematic plan view of the diaper element, having a further variant of the wavy connection regions; and

FIG. 6 shows a schematic plan view of the diaper element, having an alternative variant of the wavy connection regions.

### DETAILED DESCRIPTION

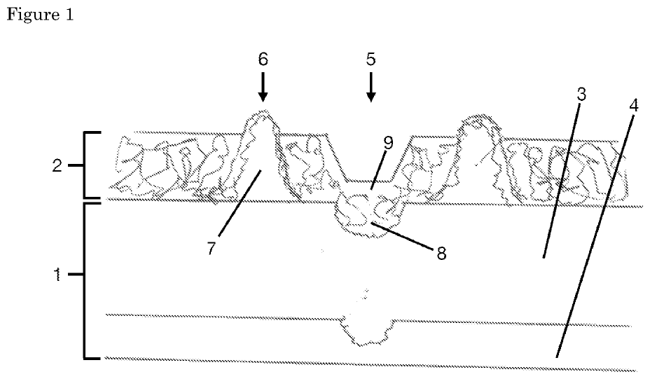

According to FIG. 1, the laminate comprises an elastic layer 1 and a non-woven layer 2 from a wavy non-woven material. This here is a hydroentangled non-woven material, wherein a spunbonded non-woven material from continuous filaments is used in the exemplary embodiment. According to the invention, the non-woven material is brought to a wavy shape prior to being connected to the elastic layer 1. Upon extruding the elastic layer 1 so as to form the wavy non-woven layer 2, the depressions of the non-woven material are impressed in the fusible elastic layer 1 such that connection regions 5 between the wavy non-woven material 2 and the elastic layer 1 are configured.

The elastic layer 1 in the exemplary embodiment is embodied as a multi-layered co-extruded film, having a core layer 3 and a further layer 4 which is configured as a skin layer. The ratio of the thickness of the core layer 3 and the further layer 4 is preferably more than 8:1, in particular more than 10:1, in particular more than 12:1. The skin layer 4 preferably has a weight between 1 and 3 g/m².

The core layer 3 is preferably composed of thermoplastic polymers. Polypropylene-polyethylene block copolymers are preferably used here, for example of the type Exxon Vistamaxx (PP based); VM 6102 or VM 6202 or VM 7810, and/or of the type Dow Infuse (PE based): Infuse 9507, Infuse 9107.

The external layer 4 is preferably composed of a polyolefin or an ethylene vinyl acetate copolymer (EVA). As opposed to the core layer 3, the external layer 4 is not sticky and thus prevents undesirable adhesion.

According to the invention, the laminate comprises connection regions 5 and reserve regions 6. The reserve regions 6 have no, or only a very weak, bond with the elastic layer 1 and preferably enclose cavities 7.

In the exemplary embodiment, the web-to-groove ratio of the roller which brings the non-woven into a wavy shape and impresses the depressions of the wavy non-woven layer 2 into the fusible elastic layer 1 is 1:6, wherein the web width is preferably 0.5 mm, and the groove width is preferably 3 mm.

The connection regions 5 according to the invention in the exemplary embodiment have different zones 8, 9.

The outer zone 9 is free of elastic material so that the elastic material does not penetrate the non-woven layer 2. The non-woven material in the outer zone 8 is not thermally influenced from the outside so that the filaments of the layer 2 from a non-woven are not fused.

A form-fitting composite of solidified elastic material and non-woven material is present in the inner zone 8 of the connection regions 5. In the exemplary embodiment here, the non-woven material is also not fused in the inner zone 8. The continuous filaments of the hydroentangled non-woven material are merely impressed into the elastic melt such that a form-fitting composite is created upon solidification. The continuous filaments of the hydroentangled non-woven material per se remain largely influenced in the connection process. These continuous filaments are merely enclosed by the fusible material of the elastic layer 1.

Upon solidification of the elastic material, there is a form-fitting composite of non-woven material and solidified material of the elastic layer 1 present in the inner zone 8.

The laminate illustrated in the figure is interconnected between a pair of rollers in which, when viewed in the drawing, a profiled roller having elevations presses the non-woven layer 2 from above into the elastic layer 1, and a counter-roller having a small surface is disposed therebelow. In the production of the laminate illustrated in FIG. 1, a cooling roller is used as the counter-roller. The cooling roller is a steel roller. The roller acting from above is a roller that is not cooled.

The rollers used for connecting are operated at a spacing, wherein a fixed spacing is set.

The laminate preferably has a specific area weight of more than 10 g/m², in particular more than 20 g/m2, preferably more than 30 g/m², and/or less than 200 g/m², in particular less than 150 g/m², preferably less than 100 g/m².

In the exemplary embodiment, the connection regions 5 and the reserve regions 6 are embodied in the shape of strips, wherein the strips run transversely to the tensile direction of the diaper element.

The strips of the connection regions 5 have a width between 0.1 and 1 mm. The connection regions 5 in the exemplary embodiments have a width of 0.5 mm.

The strips of the reserve regions 6 that do not have a connection between the non-woven layer 2 and the elastic layer 1 have a width between 2 and 6 mm. The reserve regions 6 in the exemplary embodiment have a width of 3 mm. In this way, the connection regions 5 in the exemplary embodiment have a proportion of 0.5/3.5=14.3%, and the reserve regions 6 have a proportion of 3/3.5=85.7% in terms of the surface of the flat, unstretched elastic layer 1.

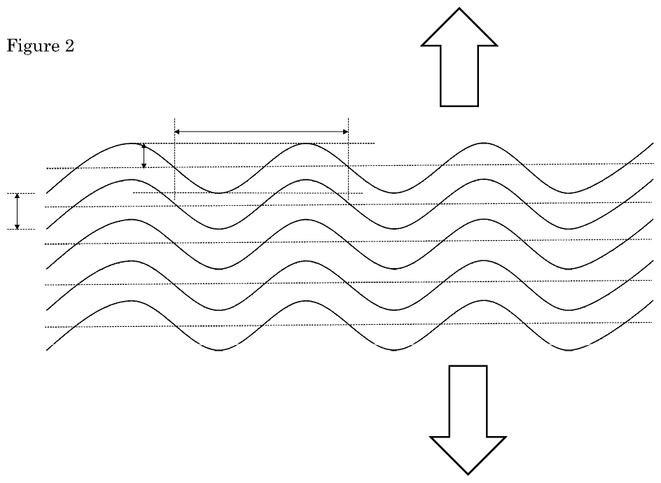

FIG. 2 shows a schematic plan view of the diaper element, having a first variant of the wavy connection regions 5. The connection regions 5 extend transversely to the tensile direction. The connection regions 5 are embodied as continuous wavy lines, wherein the spacing 10 of these lines in the exemplary embodiment is approx. 3 mm.

The imaginary auxiliary line 13 of the wavy profile of the connection regions 5 marks the deflection of the wave as the amplitude 11 in relation to the imaginary auxiliary line 13. In the regular repeating shape of the wavy profile of the connection regions 5, the length of the phase 12 marks the smallest local interval. The length of one phase 12 of the wavy line extends in the range of 30 to 100 mm. The amplitude 11 has a dimension between 1 to 6 mm. The ratio between the amplitude 11 and the phase 12 is more than 0.001, preferably more than 0.005, in particular more than 0.01, and/or less than 0.6, preferably less than 0.4, in particular less than 0.2.

The block arrows which, when viewed in the drawing, point upward and downward show the tensile direction of the diaper element under stress. The imaginary auxiliary line 13 corresponds to the direction of extent of the connection regions 5.

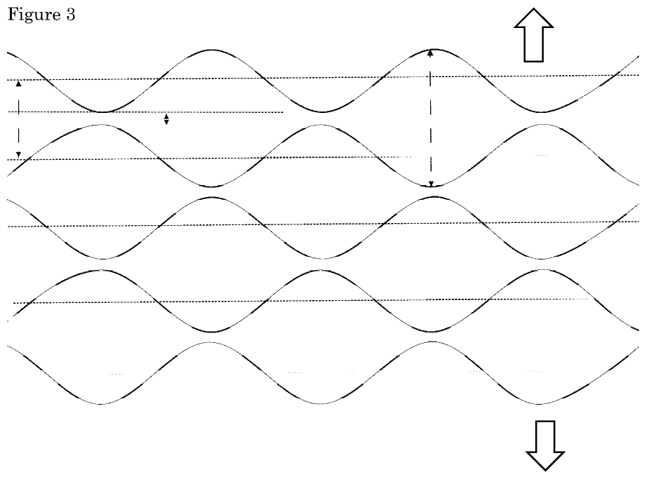

FIG. 3 shows a schematic plan view of the diaper element, having a second variant of the wavy connection regions 5. Those connection regions 5 extend along the imaginary auxiliary line 13 and thus transversely to the tensile direction of the diaper element under stress, the latter being indicated by block arrows which, when viewed in the drawing, point upward and downward.

Two connection regions 5 here are in each case mutually aligned so as to be mirror symmetrical. The spacing 14 of the imaginary auxiliary line 13 is at least double the amplitude 11 plus the minimum spacing 15, the latter being at least 1 mm. This results in a maximum spacing 16 of the connection regions 5 that corresponds to four times the amplitude 11 plus the minimum spacing 15.

Reserve regions 6 (not illustrated in the figure) in which the wavy non-woven layer by way of the elevations thereof protrudes in pleats from the central layer and encloses cavities 7 are disposed between the connection regions 5. These regions 6 serve as a reserve for stretching of the diaper element. By virtue of the connection regions 5 that extend in a wave-shaped manner, the reserve regions 6 have at their disposal different reserves of the non-woven layer and thus different reserves in terms of elongation capability, said reserves complementing one another in a mirror-symmetrical manner.

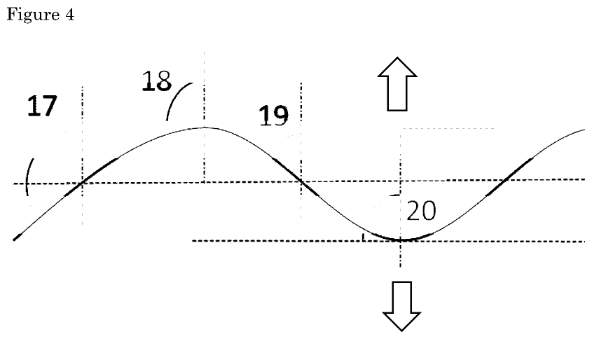

FIG. 4 schematically shows a wavy profile of a connection region 5 which extends about the imaginary auxiliary line 13 in a wave-shaped manner. The block arrows which, when viewing the drawing, point upward and downward indicate the tensile direction of the diaper element under stress.

This results in an angle between the gradient tangent of the connection region 5 and the direction of the tensile stress that continually varies in the wavy profile. In the plan view here, the angle at the wave peak 18 as well as the angle at the wave trough 20 are exactly 90°. Proceeding from an angle at the wave trough (comparable to 20) toward the angle at the wave peak 18, this angle continually increases, reaches a maximum angle 17 of 135° at the reversal point, and then continually decreases toward the angle at the wave peak 18. In the further profile of the connection region 5, the angle between the gradient tangent and the direction of the tensile stress further decreases until said angle reaches a minimum angle of 45° at the reversal point 19, from there continually increasing.

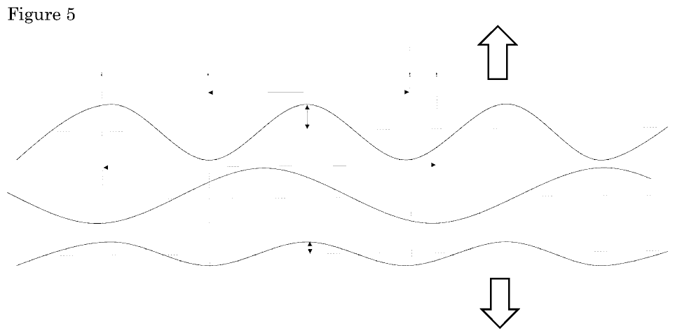

FIG. 5 shows a schematic plan view of the diaper element in a further variant of the invention in terms of the connection regions 5 that extend about the imaginary auxiliary line 13 in a wave-shaped manner. The connection regions 5 disposed next to one another may differ in terms of the amplitude 11 and 22 thereof, as well as in terms of the wavelength 12 and 21 thereof. Reserve regions 6 which provide different reserves of the non-woven layer for stretching are thus created. When viewed in detail, reserve regions 6 which appear to be random and, when viewed on a larger area, result in an orderly pattern of reserve regions that is regularly repeated, are formed.

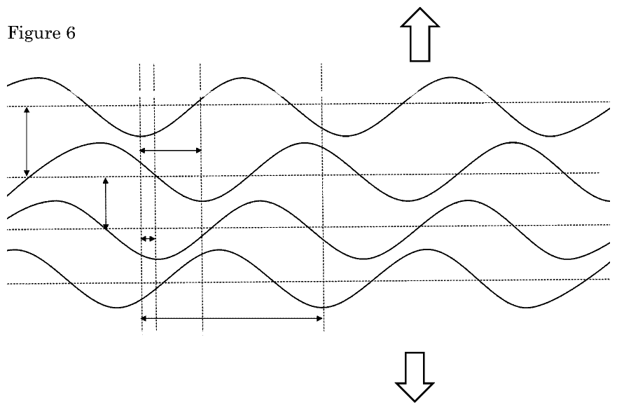

FIG. 6 shows a schematic plan view of the diaper element, having an alternative variant of the wavy connection regions 5. Those connection regions 5 extend along the imaginary auxiliary line 13 and thus transversely to the tensile direction of the diaper element under stress, this being indicated by the block arrows which, when viewed in the drawing, point upward and downward.

Connection regions 5 which are disposed next to one another here are identical in terms of the amplitude 11 and the wavelength 12 thereof, but are offset in terms of the phases thereof. This phase shift 23, in the case of connection regions 5 that are next to one another, can be implemented in the range from 0 to 2. In the case of connection regions 5 that are not disposed directly next to one another, the phase shifts 25 and 26 can be in the range from 0 to 2n.

This has the consequence that the imaginary auxiliary lines 13 may have different spacings 22 and 24 which result when the connection regions 5 do not intersect and at the same time are disposed as densely as possible. Reserve regions 6 which provide different reserves of the non-woven layer for stretching are created from this observation. In terms of a small region of this schematic plan view, reserve regions 6 which appear to be random are formed, said reserve regions 6 when viewed on a larger area resulting in an orderly pattern of reserve regions that is regularly repeated.

1. An elastic diaper element, comprising:
an elastic layer (1);
a layer (2) of a nonwoven;
connection regions (5) between the elastic layer (1) and the layer (2) of the non-woven, the connection regions (5) have an extent in a preferred direction, wherein the layer (2) of the non-woven in an unstretched state of the diaper element is wavy and provides regions (6) as a reserve for enabling stretching; and
the connection regions (5) in an extent thereof have an oscillating profile.

2. The diaper element as claimed in claim 1, wherein the connection regions (5) have a wavy profile.

3. The diaper element as claimed in claim 1, wherein or the connection regions (5) have a zigzag profile.

4. The diaper element as claimed in claim 1, wherein the extent of the connection regions (5) runs perpendicularly to as tensile direction of the diaper element.

5. The diaper element as claimed in claim 1, wherein a first phase of the oscillating profile has a length (12) of more than 10 mm and less than 200 mm.

6. The diaper element as claimed in claim 1, wherein a first phase of the oscillating profile has an amplitude (11) of more than 0.5 mm and less than 10 mm.

7. The diaper element as claimed in claim 1, wherein the layer (2) of the non-woven comprises a hydroentangled non-woven material, and the non-woven material has a specific weight of 10 to 70 g/m².

8. The diaper element as claimed in claim 1, wherein the elastic layer (1) is constructed in multiple layers and includes one core layer (3) and at least one further layer (4).

9. The diaper element as claimed in claim 1, further comprising a second layer of a non-woven, wherein the elastic layer (1) is disposed between the layer of the non-woven and the second layer of the non-woven.

10. A method for producing an elastic laminate having an elastic layer (1) and a layer (2) of a non-woven, the method comprising:
generating connection regions (5) between the elastic layer (1) and the layer (2) of the non-woven, with the connection regions (5) having an extent in a preferred direction, and the layer (2) from the non-woven, in a non-stretched state of the diaper element being formed as wavy providing regions (6) as a reserve for enabling stretching; and
the connection regions (5) are generated in a profile that oscillates in the extent of said connection regions (5).

11. The method as claimed in claim 10, further comprising generating the connection regions (5) using rollers which have flutes that run in an oscillating manner.

12. The method as claimed in claim 10, wherein the connection regions (5) are generated using mutually engaging oscillating rollers.

13. The method as claimed in claim 10, further comprising incorporating the elastic laminate in a diaper as an elastic diaper element.
